(19) United States
(12) Patent Application Publication
(10) Pub. No.: US 2025/0104822 A1
(43) Pub. Date: Mar. 27, 2025

(54) SYSTEM FOR AUTOMATICALLY GENERATING A MEDICAL SESSION REPORT AND A METHOD THEREOF

(71) Applicant: Ocean Friends Inc., Irving, TX (US)

(72) Inventors: Animesh Kumar, Irving, TX (US); Manish Shukla, North Bend, WA (US)

(21) Appl. No.: 18/474,288

(22) Filed: Sep. 26, 2023

(51) Int. Cl. G16H 15/00; G06F 40/186; G06F 40/40; G06Q 40/08; G16H 10/60

(52) U.S. Cl. CPC G16H 15/00; G06F 40/186; G06F 40/40; G06Q 40/08; G16H 10/60

(57) ABSTRACT

A system and method for automatically generating a medical session report is disclosed. The system receives patient's data and processes it using machine learning techniques and language learning models by extracting relevant information from the patient's data. The one or session note is automatically generated based patient's data, processed patient's data, and the one or more pre-stored information. The generated one or more session notes are compiled to automatically generate a medical session report of the patient. Further, the medical session report is communicated with one or more remote users including insurance companies, parents and/or a Clinician.

### TECHNICAL FIELD

The present disclosure relates to a system and related method(s) for automatically generating a medical session report. In particular, the disclosure relates to generating a medical session report for one or more sessions of a patient with a Clinician. The medical session report in the present disclosure is generated using machine learning techniques and large language models.

### BACKGROUND

Most often managing a health condition requires regular communication between a person and an expert to ensure proper guidance, support, and monitoring. Some of these conditions require therapy sessions provided by experts. For example, a person with mental health issues, like depression, anxiety, or bipolar disorder, benefit from regular therapy or counseling sessions with a mental health expert. Similarly, in rare conditions like Alzheimer's and Autism, the guardian of the person seek out support from a health professional, such as therapists or counselors.

Effective therapy sessions of the person having any of the abovementioned conditions involve comprehensive analysis and documentation of patient's progress and treatment plans. Traditionally, therapists create SOAP notes (Subjective, Objective, Assessment, and Plan) manually, which can be time-consuming and error-prone. SOAP notes refer to a common notetaking template that Clinicians use to describe a therapy session with a patient. The notes also serve as a standard template that is shared with the insurance companies for medical claims. Therefore, such therapy notes help Clinicians communicate about patient's progress in a standardized way.

The acronym SOAP stands for four sections included in the SOAP notes—Subjective, Objective, Assessment, and Plan. Together, SOAP notes provide a systematic assessment of patient's progress and interventions. SOAP notes provide a concisely written history of what a therapist observed about the patient in the session, which can also be used by insurance companies in validating insurance claims filed by the patient. Also, the structure of SOAP notes allows professionals to identify and track patient's progress. Insurance companies assess SOAP notes to substantiate billing requests and treatments.

Many healthcare fields have adopted SOAP notes as the standard format for professionals to detail an interaction with a patient to another professional in a structured way. Generally, SOAP notes represent a standard way for healthcare professionals to communicate with one another to assess, diagnose, and treat a patient. Taking precise SOAP notes that provide excellent clinical rationale elevates the therapy in the eyes of other professionals.

The accuracy of these notes greatly impacts the quality of therapy provided and can influence the therapy outcomes in longer period. Similarly, therapy planning can benefit significantly from seamless collaboration between therapists and caregivers, allowing tailored treatment plans and increased engagement throughout the therapeutic process.

Traditionally, treatment plans are in the form of written documents that outline the progression of therapy given to a patient. Treatment plans can include varied level of details and may use different representations. For example, treatment plan may include information in text, table, graph or other representations. Treatments plans are introduced in order to standardize and improve the predictability and the outcome of the prescribed treatment. For instance, the treatment plans communicate the purpose of a given treatment to all parties involved in the process including patients, relatives, referral sources and insurance companies. They provide a measure for a patient's progress in treatment and keep the entire treatment team informed of the treatment's status. Thus, problems identified at assessment level are not forgotten and appropriate follow up measures can be taken in the next therapy session. Treatment plans also allow changes and deviations to be identified and recorded so that patients and his/her caregivers are always informed with respect to the treatment steps, any changes and progression.

To obtain these desired benefits, treatment plans need to be accurate and updated whenever changes are required. This is a cumbersome and time-consuming process, which may make it difficult for the Clinicians to keep up with changes in the treatment plan. In addition, the Clinicians may skip recording changes and/or record changes with sufficient details.

Several attempts have been made to develop therapy documentation solutions and platforms. While some solutions exist for SOAP note generation, they often rely on templates, which might not capture the unique detail of each patient's case. Furthermore, most existing systems lack the capacity to accept inputs in different formats like text, images, or videos, thereby limiting the versatility and scope of therapy planning.

Moreover, the caregivers coordinate with many professionals for proper patient care. The other professionals count on previous professionals or therapists to properly document the interactions patient had with them. Failing to do so can lead to misleading or confusing information, negatively impacting the patient's overall medical care. The structured nature of SOAP notes means they can serve as a useful index to look up specific information quickly. Insurance and auditing agencies also have adopted SOAP notes to corroborate treatment claims and assess work systematically. Proper SOAP notes will show insurance providers that Clinicians are providing a skilled service that benefits the patients.

Therefore, there is a need for an improved system and method for documenting the health condition of patients. A system is needed that can reduce the overall time spent by the Clinicians in documenting the session they had with various patients each day. Furthermore, the system should be easy to use by the professionals, parents and caregivers.

### SUMMARY

The present disclosure discloses a system and method of automatically generating a medical session report of a health condition of a patient. The present disclosure addresses a need to generate SOAP notes automatically using machine learning techniques and generative AI models, and hence saves time of Clinicians that can be further used by them on the treatment of the patient.

In one aspect of the present disclosure, a method of automatically generating a medical session report for one or more sessions of a patient with a Clinician is disclosed. The method disclosed herein includes receiving the patient's data in one or more formats from one or more sources. The patient's data is processed using machine learning techniques and language learning models by extracting relevant information from the patient's data. The one or more session note is automatically generated based on the patient's data, the processed patient's data, and one or more pre-stored information. The generated one or more session notes are then compiled to automatically generate the medical session report of the patient.

In another aspect of the present disclosure, a system for automatically generating a medical session report for one or more sessions of a patient with a Clinician is disclosed. The system includes a server and a processing device that is operatively coupled to the server. The server includes a memory to store various treatment plans including previous and ongoing medical data of the patient, while the processing device executes the instructions to: receive a patient's data in one or more format from one or more sources; process the patient's data using machine learning techniques and language learning models by extracting relevant information from the patient's data; generate one or more session notes automatically based on the patient's data, the processed patient's data, and one or more pre-stored information; and compile the one or more session notes to automatically generate the medical session report of the patient.

In an embodiment of the present disclosure, the patient's data may be inputted by the Clinician, where the Clinician may provide short-hand notes using one or more prompts such as S:, O:, A:, and P:.

In yet another embodiment of the present disclosure, the patient's data be fetched from one or more other sources such as Clinician's short-hand notes from the session, audio recording of the session, video recording of the session, transcript of the session, patient's past electronic health records, past session notes, clinical assessments, patient's interviews and other relevant health condition information.

Advantageously, the user interface may include a graphical user interface, a voice user interface, a gesture user interface, or any suitable user interface to receive and share the patient's data and medical session report, respectively. Also, the user may provide a text input, a voice command, a video, or a gesture as an input.

### BRIEF DESCRIPTION OF THE ACCOMPANYING DRAWING

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

FIG. 1 illustrates a computer implemented system used for automatically generating a medical session report for one or more sessions of a patient with a Clinician, in accordance with one implementation of the present disclosure.

FIG. 2 depicts an exemplary implementation disclosing working of a data model for automatically generating a medical session report for one or more sessions of a patient with a Clinician, in accordance with one implementation of the present disclosure.

FIG. 3 depicts an exemplary implementation disclosing the working of a data model for generating a SOAP notes, in accordance with one implementation of the present disclosure.

FIG. 4 illustrates a method for automatically generating a medical session report for one or more sessions of a patient with a Clinician, in accordance with one implementation of the present disclosure.

FIG. 5 illustrates an exemplary method for automatically generating a medical session report of a patient by preparing SOAP notes and sharing it with the insurance or billing companies, in accordance with one implementation of the present disclosure.

FIG. 6a illustrates an exemplary view of a dashboard showing patient's details and goal selection for automatically generating a medical session report of the patient, in accordance with one implementation of the present disclosure.

FIG. 6b illustrates an exemplary view of the dashboard showing the details inputted by a Clinician and details of SOAP notes prepared on different dates for automatically generating a medical session report of a patient, in accordance with one implementation of the present disclosure.

FIG. 7a illustrates an exemplary view of the dashboard showing a process of editing the SOAP notes inputted by the Clinician on the dashboard, which includes therapy details and billing information, in accordance with one implementation of the present disclosure.

FIG. 7b illustrates an exemplary view of the dashboard showing the process of editing the SOAP notes inputted by the Clinician on the dashboard, which includes the subjective and objective aspect (long-term goals) of the SOAP notes, in accordance with one implementation of the present disclosure.

FIG. 7c illustrates an exemplary view of the dashboard showing the process of editing the SOAP notes inputted by the Clinician on the dashboard, which includes the objective aspect (short-term goals) of the SOAP notes, in accordance with one implementation of the present disclosure.

FIG. 7d illustrates an exemplary view of the dashboard showing the process of editing the SOAP notes inputted by the Clinician on the dashboard, which includes the assessment aspect of the SOAP notes, in accordance with one implementation of the present disclosure.

FIG. 7e illustrates an exemplary view of the dashboard showing the process of editing the SOAP notes inputted by the Clinician on the dashboard, which includes the planning and home-based exercise aspect of the SOAP notes, in accordance with one implementation of the present disclosure.

FIG. 8 illustrates an exemplary graphical representation of the progress made by the patient after a few therapy sessions, in accordance with one implementation of the present disclosure.

### DETAILED DESCRIPTION

Embodiments, of the present disclosure, will now be described with reference to the accompanying drawing.

In the following description, certain specific details are set forth in order to provide a thorough understanding of various disclosed embodiments. However, one skilled in the relevant art will recognize that embodiments may be practiced without one or more of these specific details, or with other methods, components, materials, etc.

Unless the context indicates otherwise, throughout the specification and claims which follow, the word “comprises” and variations thereof, such as, “comprises” and “comprising” are to be construed in an open, inclusive sense that is as “including, but not limited to.” Further, the terms “first,” “second,” and similar indicators of the sequence are to be construed as interchangeable unless the context clearly dictates otherwise.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

The Subjective, Objective, Assessment, and Plan (SOAP) note is a commonly used note taking template across healthcare field, so that the Clinician can easily standardize the note-taking process and share it with other practitioners, billing/insurance companies, parents or caregivers, if needed. Each note follows the same structure, and all the information is clearly laid out in different sections for the parent/caregivers, billing/insurance companies or any other Clinician. This helps the parents/caregivers to know exactly what is going on in a child's treatment plan and what is the progress made by the child. Further, if the parents/caregivers switch the therapist or is undergoing any other therapy for their child, then another Clinician will get to know what happened in the previous therapy sessions.

While the description presented herein may makes certain references to Autism, it is to be appreciated that aspects of the present disclosure are also equally applicable to other conditions such as Dementia, Alzheimer's, Parkinson, or any other health condition.

The system and method disclosed herein includes receiving the patient's data in one or more formats from one or more sources. The patient's data include notes taken up by a Clinician which is inputted into the user interface. These notes can be inputted by the Clinician in any format like text, video, audio, image, recording from the session and so on. The one or more sources from where the patient's data is received may include patient's medical history (electronic health record, etc.), previously generated SOAP notes (in previous sessions), session notes generated by the Clinician in the ongoing session, and so on. The patient's data is processed using machine learning techniques and language learning models by extracting relevant information from the patient's data. The one or more session notes are automatically generated based on the patient's data, the processed patient's data and one or more pre-stored information. In particular, the patient's data inputted by the Clinician is populated with one or more pre-stored information. The generated session notes are compiled to automatically generate a medical session report of the patient. In some instance, the generated medical session report includes one or more recommendation related to the prognosis and therapy of the patient. Further, the medical session report is communicated with one or more remote users including one or more of insurance companies, parents of the patient and/or a Clinician.

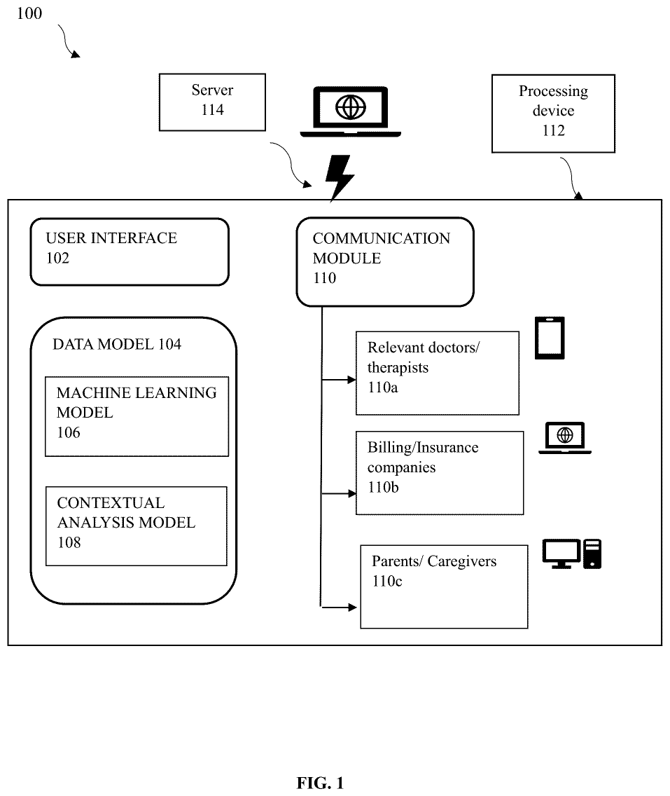

FIG. 1 illustrates a computer implemented system 100 used for automatically generating a medical session report for one or more sessions of a patient with a Clinician, in accordance with one implementation of the present disclosure. While the system 100 discussed here refers to automatically generating a medical session report of a patient, it should be noted that the system 100 may also be used for communication purpose, but is not limited to, seamless communication between Clinician and parent/caregiver, providing details to billing/insurance companies and the like.

The system 100 includes one or more components that work in tandem to provide a seamless user experience. In the shown embodiment, the system 100 includes a processing device 112 that is communicatively coupled to a server 114. As shown, the processing device 112 includes a user interface 102, a data model 104, a communication module 110, while the server 114 includes a memory (not shown in the FIG. 1) which may store the details of the patient's past medical record, details of previously generated SOAP notes, details of one or more session notes from the ongoing sessions, and so on. The data model 104 further includes a machine learning model 106 and a contextual analysis module 108 for processing and automatically generating the SOAP notes from the patient's data inputted by the Clinician. The communication module 110 is configured to communicate the automatically generated SOAP notes to one or more remote users, which may include relevant doctors/therapist 110a, billing/insurance companies 110b, parents/caregivers 110c and so on.

The processing device 112 serves as a primary interface between the Clinician and the system 100. The user interface 102, data model 104 and communication model 110 are operatively coupled to each other. The user interface 102 is designed to be intuitive and easy to use, enabling the Clinician to input patient's data without any difficulty. The user interface 102 is configured to receive the input from the Clinician in various formats, for instance, the input provided by the Clinician in the user interface 102 may be in the form of text input, audio input, video input, recording of a session, image input or a combination of any of these. In some scenarios, the Clinician input may be related to progress of the health condition of the patient. However, in other scenarios, the Clinician input may be related to any information that is not directly related to progress of the health condition of the patient. For example, it may include the details for billing companies, or at what time the child reached the therapy center, and so on.

The system 100 disclosed herein allows the Clinician to provide inputs through the commonly used user interfaces 102 which may include a graphical user interface, a voice user interface, a video user interface, a gesture user interface, or any suitable user interface. In an implementation, the Clinician may provide an image input or video input of the child to the user interface 102. Based on that input the data model 104 will process the input data and automatically generate SOAP notes using the current input details and the previous medical record of the child and the SOAP notes that are generated by the Clinician in the previous sessions. Similarly, a Clinician may record the whole therapy session and use it as an input in order to automatically generate the SOAP notes using data model.

The user interface 102 is operatively coupled to the data model 104 to receive patient's data from Clinician in a seamless manner. In an example, the Clinician may simply access the user interface 102 and start writing (text input) details of the patient in a very simple language. For example, ‘if the child came to the therapy center along with his father and his father reported that the child did not sleep well last night’, the Clinician may provide a text input to the user interface 102 as “the child bd and did not sleep well last night”.

The system 100 allows contextual analysis of the Clinician inputs using fine-tuned large language models and machine learning techniques. Such analysis allows extraction and categorization of the input data provided by the Clinician on the basis of defined categories like Subjective, Objective, Assessment and Planning defined under SOAP notes. The contextual analysis module 108 make use of machine learning module 106 in comparing the ongoing patient's data with the past generated SOAP notes of the patient stored in the repository in order to find which input falls under which category. Further details pertaining to the categorization of the patient's data to generate a session note divided into various categories (Subjective, Objective, Assessment and Planning) will be discussed in subsequent section.

The processing device 112 of the system 100 can be any device that is equipped with the necessary components to interact with the Clinician, receive inputs in the form of patient's data, process patient's data using data model 104, generate a session note in correspondence with patient's data and communicate the medical session report with one or more remote users like relevant Clinician 110a, billing/insurance companies 110b, parents/caregivers 110c and so on using a communication module 110. Some examples of such devices include smartphones, tablets, personal computers, and so on. With its advanced technology and user-friendly user interface 102, the processing device 112 can provide an ideal platform for the Clinician to interact with the system 100 and automatically generate the SOAP notes.

The data model 104 is operatively coupled with the user interface 102 and the communication module 110. It further consists of machine learning module 106 and contextual analysis module 108. The data model 104 refers to the part of the system 100 which is responsible for processing the inputs received from the Clinician. The machine learning module 106 present in the data model 104 is configured to process the patient's data inputted by the Clinician using machine learning techniques and language learning models by identifying the relevant information from one or more sources. The one or more sources may include Clinician's short-hand notes from the session, audio recording of the session, video recording of the session, transcript of the session, patient's past electronic health records, past sessions generated SOAP notes, clinical assessments, user inputs, patient's interviews and/or other relevant health condition information. The one or more session notes are automatically generated based on the patient's data, processed patient's data and one or more pre-stored information. For example, the session notes may be generated based on a correlation between the patient's data and the one or more pre-stored information. The generated one or more session notes are compiled to automatically generate a medical session report of the patient i.e., the SOAP note. In some instance, the one or more recommendation may relate to prognosis or therapy of the patient.

The machine learning module 106 compares the input data from the Clinician with the data stored in the memory, which may include past electronic health records of the user, SOAP notes generated in the past sessions, and so on. The one or more pre-stored information may include the subject patient's data, another patient's data with similar condition, and/or information obtained from a database.

The data model 104 receives the input data in the form of patient's data from the Clinician in one or more formats from one or more sources. The data model 104 is configured to identify the intent behind the data input. To that end, the data model 104 further includes a contextual analysis module 108 for analyzing the received input from the Clinician. The contextual analysis module 108 is configured to extract condition related information from the received inputs, which aid in understanding what the Clinician is trying to convey. In an example, if a Clinician inputs that “Peter came to therapy center along with mom and he was happy.” The contextual analysis module 108 will automatically classify it into the Subjective category and will further automatically generate the SOAP notes using machine learning module 106.

When generating an automatic SOAP note corresponding to the inputs received by the Clinician, the data model 104 may use a combination of factors, such as the Clinician inputs in the ongoing session, his/her previous medical data, patient's SOAP notes generated in the past sessions and so on, in order to automatically generate the SOAP notes that is more appropriate and contextual with the Clinician inputs. However, if the Clinician input is crude or inappropriate, the contextual analysis model 108 may choose to disregard that input or provide a response that encourages the Clinician to rephrase his/her input in a more appropriate manner.

The data model 104 receives the patient's data from the one or more sources which may include patient's past electronic health records, past sessions SOAP notes, clinical assessments, user inputs, patient's interviews and/or other relevant health condition information. This may involve use of machine learning algorithms or other forms of artificial intelligence to analyze the inputs, access relevant databases and knowledge sources, and generate an appropriate response. The data model 104 is a crucial component of the system 100, as it determines the behavior of the user interface 102 and the quality of the automatically generated SOAP notes that it provides to the one or more remotes users. The data model 104 should be designed to provide accurate, relevant, and helpful suggestions to the Clinician in real-time. In essence, the data model 104 is also responsible for classifying the appropriate category of the SOAP notes i.e., subjective, objective, assessment and planning, for example, if the user enters that “child was brought by dad and did not sleep well last night”. It will automatically be classified into Subjective category by the data model 104. The data model 104 disclosed herein is scalable, efficient, and robust, so that it can handle a large number of inputs and provide quick and accurate responses to the Clinician.

Further, the communication module 110 enables the processing device 112 to communicate with one or more remote users which may include relevant Clinician 110a, billing/insurance companies 110b, parents/caregivers 110c and so on. The communication module 110 is operatively coupled to the user interface 102. The communication module 110 ensures that the processing device 112 can receive and transmit data from and to the computer, mobile, tablet etc. of the one or more remote users, thereby providing the one or more remote users the actual status of the progress of the child undergoing therapy.

The SOAP notes may be communicated to a second Clinician 110a, where the patient may consult the second Clinician for additional therapy session. In an exemplary scenario, if a child has Autism and he is undergoing speech therapy in the morning and activity related therapy in the evening. The SOAP notes generated by the speech therapist may be used by the activity therapist and vice versa. In an example, the activity therapist suggested some diet related changes say, ‘to add 100 gm protein in diet’ in the planning section of the SOAP notes. The speech therapist may not have the details about the diet of the child but the speech therapist can have a look at the SOAP notes generated by the activity therapist and will look at the diet of the child accordingly. If the child mentions during the therapy assessment with the therapist that ‘he took tofu last night.’ The speech therapist will mention it under the ‘Subjective’ category of the SOAP notes and the quantity of proteins will also get mentioned in the SOAP notes using data model 104. It will become easy for the speech therapist to know the exact amount of protein, fat, carbohydrates intake by the child, which might be difficult for him/her to understand without the data model 104.

Further, the billing/insurance companies 110b may use the communicated SOAP notes for settling the insurance claim submitted by the family of the child. Since, proper documentation is very necessary for getting the insurance claims approved. Further, the therapy details communicated with the parents/caregivers 110c may help them to continue the therapy procedures at home also, so that child gets extra benefits and improves his/her progress. Also, the parents/caregivers will get to know about the status of the progress of the health of the child using the SOAP notes shared with them.

To that end, the communication module 110 should be designed to be flexible and adaptable, allowing the user interface 102 to communicate the SOAP notes containing the progress details of the health condition of the patient in the format that is most convenient for the user, and in a way that is easily accessible and understandable. The communication module 110 should also be designed to be secure, ensuring that the patient's information and data remain confidential and protected.

In this scenario, the processing device 112 communicates with the server 114 via a communication network. The server 114 acts as the backbone of the system 100, which stores the documents related to the progress of the health conditions of the patient in the form of SOAP notes along with inputs provided by the Clinician and previously generated SOAP notes and past electronic medical records of the patient in the memory. Also, in this scenario, a user profile is created whenever the Clinician creates a new and unique profile of a new patient and interacts with the user interface 102 for a first time. The user profile includes information related to the patient's health conditions, device related information, Clinician inputs, billing/insurance companies' details and generated SOAP notes. It may also be noted that in such scenario, the all such information is stored in the memory for any future use.

The system 100 for documenting the progress of health condition of a patient is designed to provide individuals with an effective way to check the progress of their condition and automatically generate a SOAP note. The natural language processing technology used by the system 100 ensures that the Clinician inputs are understood and processed accurately, thereby generating an appropriate SOAP note.

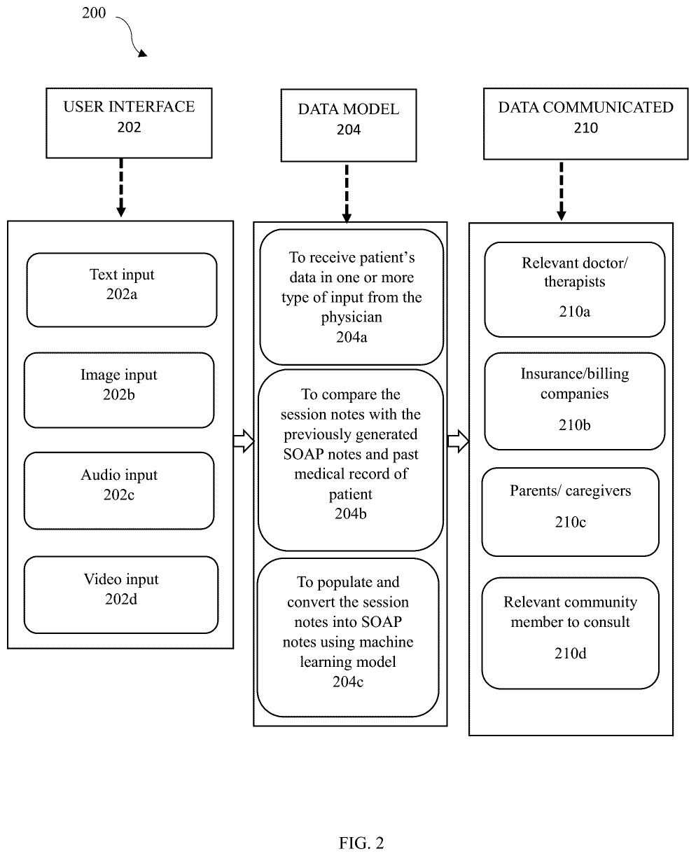

FIG. 2 depicts an example implementation 200 disclosing the working of the data model for automatically generating a medical session report for one or more sessions of a patient with a Clinician, in accordance with one implementation of the present disclosure. In comparison to FIG. 1, here the detailed use of user interface 202, data model 204 and communication module is shown.

As discussed previously, the Clinician enters the patient's data related to the patient's health condition is received by the user interface 202. The patient's data is provided via the user interface 202 which may be in the form of text input 202a, image input 202b, audio input 202c, video input 202d or a combination of any of these. The received input data is then provided to the data model 204 for further processing. The data model 204 processes the inputted data to classify the patient's data into one or more categories pertaining to SOAP notes. This helps in automatic generation of the SOAP notes based on their different categories i.e., Subjective, Objective, Assessment and Planning. More specifically, the data model 204 receives the patient's data in one or more formats from the one or more sources and enter it into the user interface 202 204a. The one or more sources may include Clinician's short-hand notes from the session, audio recording of the session, video recording of the session, transcript of the session, patient's past electronic health records, past sessions SOAP notes, clinical assessments, user inputs, patient's interviews and/or other relevant health condition information. The machine learning module then processes the patient's data inputted by Clinician by comparing and identifying the relevant information from the one or more sources 204b. The session note is automatically generated in correspondence with the patient's data by populating the at least one or more medical notes of Clinician with one or more pre-stored information. The session notes are generated based on a correlation between the patient's data and the one or more pre-stored information. The generated session notes are compiled to automatically generate a medical session report of the patient. In some cases, the medical session report may include one or more recommendation related to the prognosis and therapy of the patient 204c.

The SOAP notes are then communicated 210 to one or more remote users which may include relevant Clinician 210a, insurance/billing companies 210b, parents/caregivers 210c, relevant community member for consultation 210d.

Similar to above, the data model 204 analyzes each input and interpret if the input should be stored under which category like Subjective, Objective, Assessment and Planning.

Further, the data model 204 is configured to handle different formats of data, including text, audio, image, recording of the session, video and so on. Also, the data model 204 can handle unstructured data. The data model 204 can be a machine learning model and may be configured to process and analyze fed data using techniques such as language learning models and NLP (natural language processing). The data model 204 may also define the input and output format of the data and how it is processed by the machine learning algorithm. In an exemplary scenario, if the Clinician wishes to enter the details of subjective category of the SOAP notes, he/she may write it in either of the two ways: 1. may start with a prompt ‘S’, here S denotes for subjective. Similarly, O, A and P for objective, assessment and planning respectively. 2. may directly start inputting the data without mentioning any initial prompt. In an implementation, the Clinician may input the data in the form of “S: Alex came along with dad and didn't sleep last night” and in another implementation, the physician/therapist may input the data “Alex came along with dad and didn't sleep last night.” The contextual analysis module present in the data model 204 of the system 100 will automatically categorize the context into its specific categories based on the input provided by the Clinician. It also uses the machine learning module, which may learn from SOAP notes of previous sessions. The machine learning module compares the input data with previous notes and classifies the inputted data in one or more SOAP notes categories (e.g., S—Subjective, O—Objective, A—Assessment, and P—Plan_accordingly.

Further, the data model 204 can learn and improve over time, as it gets exposed to more inputs by Clinician, response, and other related data. The data model 204 is trained using large amounts of data and sophisticated machine learning techniques, which enables the data model 204 to learn patterns and relationships within the data. Further, the data model 204 gets updated each time the Clinician provides a new input and a corresponding SOAP note is generated.

The data model 204 disclosed is configured to learn and adapt to new inputs and feedback from Clinician. In an example, if the patient consistently provides positive response for certain goals set up by the Clinician in the Objective category, the data model 204 may learn to prioritize those options in the future and set up them in the planning category, as they are helping in child's progress. Similarly, if patient consistently provide negative for certain goals set up by the Clinician in the Objective category, the data 204 model may learn to avoid those options in the future, as the child id not responding to those treatment techniques.

Exemplary data models may include various Generative Artificial Intelligence approach which may include GPT-3 (Generative Pre-trained Transformer 3), GPT-3.5, BERT (Bidirectional Encoder Representations from Transformers), RoBERTa (Robustly Optimized BERT Approach), ALBERT (A Lite BERT), T5 (Text-to-Text Transfer Transformer), or any other suitable models known to those skilled in the art.

In summary, data model 204 can handle different types of input data, including images and sound, along with text, as long as the structure and relationships between the data is properly defined in the data model 204.

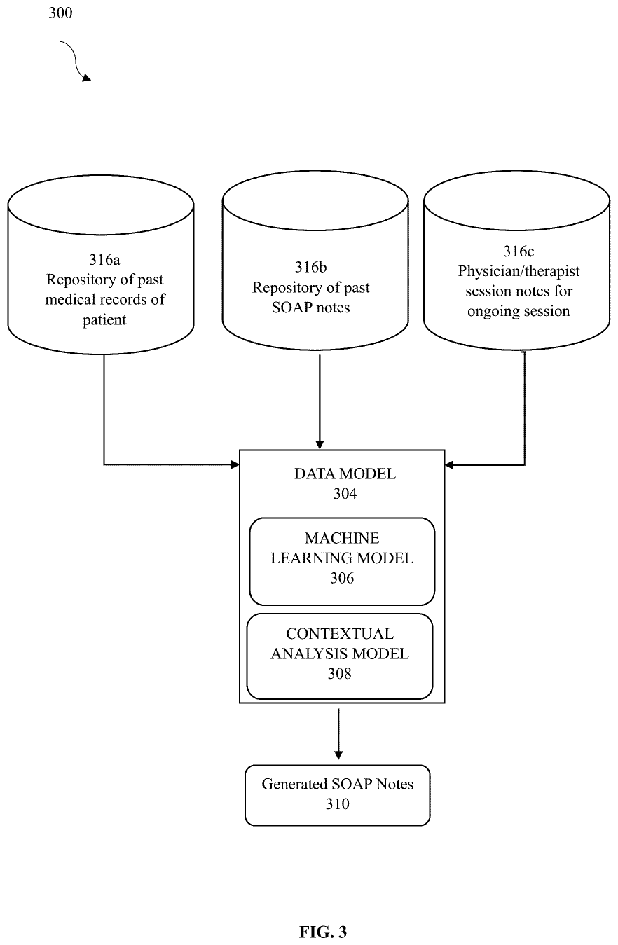

FIG. 3 depicts an example implementation 300 disclosing the working of the data model and the use of memory along with the data model for generating the SOAP notes, in accordance with one implementation of the present disclosure.

It discloses the working of the data model 304 i.e., using different types of input sources and other details to generate a SOAP note. The data is provided to the data model 304 using one or more sources stored in the repository. The one or more sources may include repository of past medical health record of patient 316a, repository of past SOAP notes generated in the past held sessions 316b and the at least one or more session notes of the ongoing session 316c. The at least one or more session notes of the ongoing session 316c includes clinical assessments, user inputs, patient's interviews and so on. All the above-mentioned data is provided to the data model 304. In addition, the one or more sources may include Clinician's short-hand notes from the session, audio recording of the session, video recording of the session, and transcript of the session.

The data model 304 consists of a machine learning module 306 and a contextual analysis module 308. The data model 304 uses machine learning module 306 and contextual analysis module 308 to process the patient's data inputted by the Clinician and is further configured to automatically generate the SOAP notes. The machine learning module 306 is configured to process the patient's data inputted by Clinician in the ongoing session using machine learning techniques and language learning models by identifying the relevant information from the one or more sources. The contextual analysis module 308 is configured to communicatively operate with the machine learning module 306 and generate a detailed version of SOAP notes automatically by populating the patient's data of Clinician with one or more pre-defined information in order to generate a session note. The one or more pre-defined information includes patient's data, another patient's data with similar condition and/or information obtained from a database. The session notes are generated based on a correlation between the patient's data and the one or more pre-stored information. The generated session notes are compiled to automatically generate a medical session report of the patient which includes one or more recommendation related to the prognosis and therapy of the patient. Further, the medical session report is communicated with one or more remote users including one or more of insurance companies, parents of the patient and/or a Clinician.

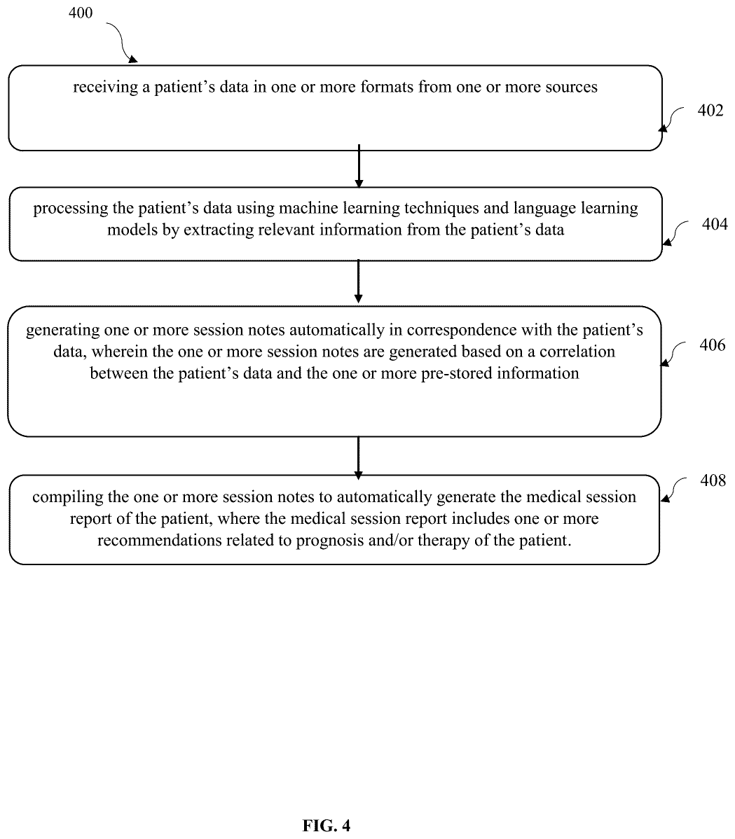

FIG. 4 illustrates a method for automatically generating a medical session report of a patient, in accordance with one implementation of the present disclosure.

The method 400 for automatically generating a medical session report of a patient includes:

Step 402 involves receiving a patient's data in one or more format from one or more sources. The one or more sources may include Clinician's short-hand notes from the session, audio recording of the session, video recording of the session, transcript of the session, patient's past electronic health records, past sessions SOAP notes, clinical assessments, Clinician inputs, patient's interviews and/or other relevant health condition information. The input provided by the Clinician can be in any format like text input, audio input, image input, video input, recording from the session or a combination of any of these. The patient's data include one or more notes provided by the Clinician in case the input is text.

Step 404 involves processing the patient's data using machine learning techniques and language learning models by identifying the relevant information from the patient's data. The relevant information from the one or more sources i.e., Clinician's short-hand notes from the session, audio recording of the session, video recording of the session, transcript of the session past medical record of the patient, past generated SOAP notes and so on is captured by the machine learning module and used for the processing of the patient's data.

Step 406 involves generating one or more session notes automatically based on the patient's data, the processed patient's data, and one or more pre-stored information. The pre-stored information may include patient' data or another patient's data with similar condition and/or information from a database. Here, the database may include a repository of information obtained from previous sessions of the patient or other patients. This may include the information provided by the patient about the condition or the recommendations made by the Clinician about the prognosis and therapy of various patients. Typically, the database is the repository of patients records that include all the information about patients. This information is may be generated by Clinicians or health care professionals based upon a direct or indirect interaction with the patients or individuals having knowledge of the patient's condition. Some exemplary databases may include clinical data repository (CDR) or clinical data warehouse (CDW), electronic health records (EHRs).

Further, generating the session notes automatically based on the pre-stored information may include use of generative AI models (such as large language models or LLMs like ChatGPT, Bard, etc.) and/or use of one or more relevant machine learning modules. For instance, the generative AI may use the pre-stored information to generate one or more session notes. To that ends, the generative AI model may correlate the received patient's data and correlate the same with the data stored in the database to generate a more comprehensive and accurate session note. For example, the generative AI model may consider the prognosis of the past medical session reports to generate the session notes of ongoing session. It should be noted that the generative AI model may include one or more fine-tuned large language models including ChatGPT, Bard, and others. In another instance, the correlation of patient's data with the pre-stored information may be done using a machine learning module. Here, the machine learning module may identify the patterns from the patient records stored in the database to generate relevant session notes.

The contextual analysis module in communication with machine learning module categorizes the patient's data in the respective categories of the SOAP notes i.e., Subjective, Objective, Assessment and Planning. The contextual analysis module uses SOAP notes generated in the previous sessions which are stored in the machine learning model for categorizing the patient's data of the ongoing session in their respective category.

Step 408 involves compiling the session notes to automatically generate a medical session report i.e., SOAP note of patient in need.

Further the medical session report is communicated with one or more remote users, which may include insurance or billing companies, caregivers of the patient and/or other Clinician. The SOAP notes provided to the billing/insurance companies may be used by them for initiating the therapy reimbursements to the patient. The SOAP notes provided to the parents/caregivers helps in managing the level of communication between the patient's family and the Clinician in order to provide the continuity in treatment. In some instances, the method may automatically generate an insurance claim that is then submitted to the insurance companies. The present disclosure generates the insurance claim by automatically filling one or more patient and session related details in a required claim format. In addition, the method allows submission of one or medical session reports along with the insurance claim, if required.

The method 400 further involves selecting a template in correspondence to the generated one or more session notes of the patient. There are various templates pre-stored in the machine learning module which are used by the data model when the Clinician inputs the patient's data in order to automatically generate a SOAP note. The one or more selected session notes are compiled in the selected template to automatically generate a medical session report. The selected pre-stored template is in correspondence with the generated session notes of the patient. The medical data standard is decided by AMA (American Medical Association) and is followed by each and every individual belonging to that field. The patient's past electronic health records are analyzed and adapted in the session notes in order to enhance the specificity and relevance of the generated at least one or more medical notes.

In an exemplary scenario, if a patient is undergoing speech therapy, as soon as the Clinician starts inputting the patient's data. The data model will analyze the patient's data and may use the pre-stored template of the medical session report (if the patient has attended one or more therapy sessions previously) or the pre-stored template of some other patient dealing with similar condition. Similarly, if a patient comes for a therapy or consultation for weight loss treatment for the first time. When the Clinician inputs the patient's data, the data model will analyze the patient's data and may auto-generate a new template by merging two or more pre-stored templates depending on what all fields like name of patient, therapy name, and so on needs to be inserted on the medical session report.

The method 400 further allows automatic selection of one or more goals, which may include one or more long-term goals and/or one or more short-term goals. The one or more short term goals are generally the sub categories made under the respective one or more long-term goals. In present disclosure, the machine learning module automatically selects one or more long-term goals with or without the corresponding short-term goals. The automatic selection of goals is typically based on the patient's data provided by the Clinician. For example, the long-term goal selected by the module may be “Martha will sit continuously for 20 minutes and recite a poem to all her friends and family”. Based on this, the one or more short-term goals may automatically be generated by the machine learning module as “Martha will sit continuously for 7 minutes”, “Martha will recite a poem to her therapist” and so on. The one or more automatically generated short-term and long-term goals may vary, and this is just an example. The long-term goals last for a specific time period, say 6 months, which is more that the duration of the one or more short-term goals, say 2 months. The duration of the one or more long-term and short-term goals may vary depending on the response of the child towards that therapy, for instance, if the child is responding in a positive way and is finishing her goals before the allotted time, the machine learning module based on the input provided by the Clinician may change the goals to another level. Similarly, in case of negative response provided by a child towards a goal, the machine learning module based on the input provided by the Clinician will automatically change the one or more goals of the patient.

Based on the one or more automatically generated one or more short-term and long-term goals, the medical session report i.e., the SOAP notes will be generated automatically and the therapy sessions of the patient will be conducted based on these session notes.

The method 400 further allows automatic goal mapping to track the progress made by the patient on the selected one or more goals. In an exemplary scenario, a child is undergoing a therapy for speech and motion. Based on the input provided by the Clinician, the machine learning module will automatically generate one or more long-term and short-term goals for the patient. The long-term goals include “Child will recite poem in front of her friends and family and will sit continuously at a place for 20 minutes while reciting the poem”. The short-term goals based on the long-term goals may include “Child will recite a poem with cues”, “Child will sit continuously at a place for 10 minutes without misbehaving” and so on. As soon as the therapy session of the child starts, the one or more goals gets automatically selected based on the patient's data provided by the Clinician. The machine learning module using the contextual analysis module automatically maps the inputted patient's data to the corresponding goal. In particular, the module does a contextual analysis to map the input to the relevant goal and thereby automatically update the progress made by the patient against the said goal. It provides an advantage that the Clinician does not have to continuously select the goal and then update the progress made on that goal. It saves a lot of time of the therapist which can be included in providing therapy to the child. For example, ‘if the child has recited two lines of a poem and sat for 3 minutes on first day of therapy’. The data model by making use of machine learning module and contextual analysis module automatically start goal mapping and updating the progress status of the child. The data model will automatically select the respective goals based on the input and update the progress status of the child in ‘speech related goals’ and ‘motion related goals’.

The progress of the child is available on the platform which can be accessed by one or more remote users which may include parents of the patient, Clinician of the child and so on.

The progress of the child may be available to one or more remote users in the form of visual representations like graphs and charts which makes it easy and convenient for the one or more remote users to access and understand the progress made by the child.

It should be noted that the user profile of each patient is unique and exclusive to the patient. The present disclosure uses user profile of a large population of patients to provide an effective way of documenting the progress of health condition of the patient suffering from such rare condition. Further, the user profile is updated each time the Clinician provides at least one input.

In this manner, the system with access to the user profile of various patients improves the ability of the user to successfully access and receive the information and solution regarding autism based on conditions suitable for the patient. The present disclosure also provides a convenient way for users who are trying to retrieve or post details regarding specific episodes of the condition in the patients.

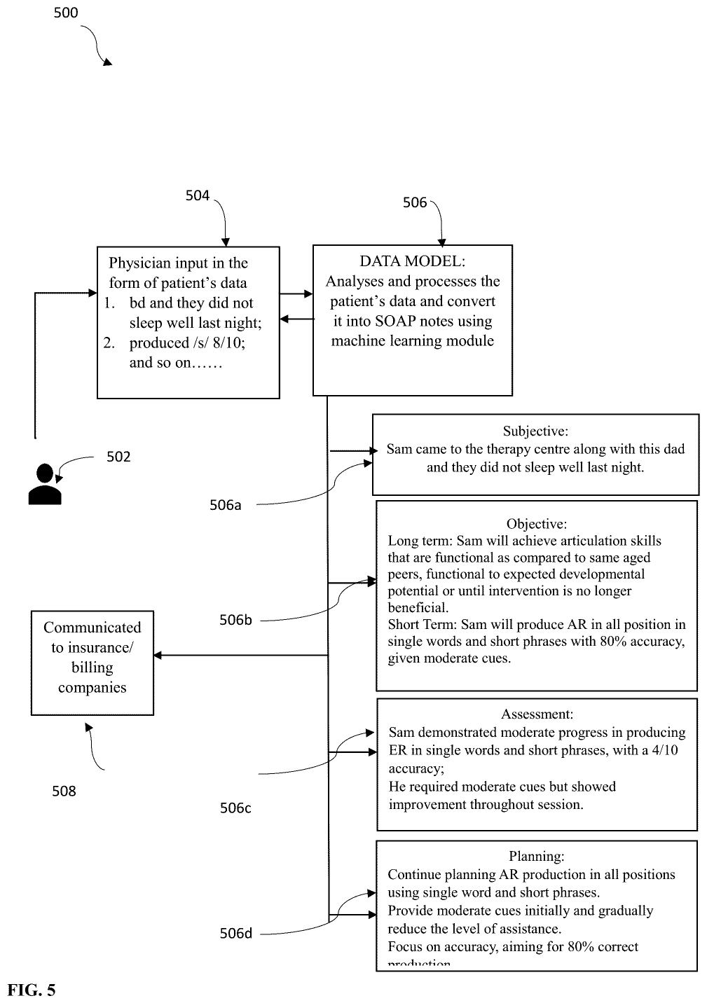

FIG. 5 illustrates an example method 500 for automatically generating a medical session report of a patient by preparing the SOAP notes and passing it on to the insurance or billing companies, in accordance with one implementation of the present disclosure.

It should be noted that the Clinician 502 who is providing the input may be the patient's physician, therapist, doctor, nurse or any other person belonging to the same field. It may be understood that the term ‘Clinician’ may be interpreted as doctor, therapist, physician, Clinician, nurse or any other related person in all other embodiments of this disclosure.

Considering that the Clinician 502 is providing the input data in the form of patient's data 504 in the user interface. In the exemplary scenario, the Clinician 502 inputted “bd and they did not sleep well last night”. Further, the Clinician 502 after starting the therapy observed the patient and inputted “produced /s/ 8/10” and so on. The data model 506 which is operatively coupled to the user interface, i.e., the place where the Clinician 502 inputs the data. The data model uses machine learning model and produces an enhanced, clearer and more detailed version of the patient's data inputted by the Clinician 502 in the form of session notes 504. The machine learning module along with the contextual analysis module processes the patient's data and generates a detailed session note without any manual intervention. The contextual analysis module classifies the input data from the Clinician 502 in categories of Subjective, Objective, Assessment and Planning (if the initial S:, O:, A:, P:) is not provided by the Clinician 502.

The data model 506 in communication with the user interface produces the following enhanced and detailed version of the patient's data in the form of one or more session notes using machine learning module and contextual analysis module.
“Subjective: Sam came to the therapy centre along with this dad and they did not sleep well last night 506a.
Objective: Long term: Sam will achieve articulation skills that are functional as compared to same aged peers, functional to expected developmental potential or until intervention is no longer beneficial. Short Term: Sam will produce AR in all position in single words and short phrases with 80% accuracy, given moderate cues 506b.
Assessment: 1. Sam demonstrated moderate progress in producing ER in single words and short phrases, with a 4/10 accuracy;
2. He required moderate cues but showed improvement throughout session 506c.
Planning: 1. Continue planning AR production in all positions using single word and short phrases.
2. Provide moderate cues initially and gradually reduce the level of assistance.
3. Focus on accuracy, aiming for 80% correct” 506d.

Based on the two lines input given by the Clinician 502, the data model 506 produces such an enhanced and detailed version of the one or more session notes. It helps in saving time of the Clinician 502 and that time can be utilized for further therapy sessions. The Clinician 502 do not have to consult the past medical records of the patient and past SOAP notes to make the SOAP notes for the ongoing session, as the machine learning module automatically generates the session notes for the ongoing session by comparing the at least one or more session notes of the ongoing session with the past medical record of the patient and the SOAP notes generated in the past sessions. Hence, this reduces the documentation time of the Clinician 502.

Further, the generated session notes are compiled to automatically generate a SOAP note based one or more recommendations related to the prognosis and therapy of the patient. The automatically generated SOAP notes are communicated to the billing/insurance companies 508 for their pursual and performing the reimbursement of the patient.

This is an exemplary description of the SOAP notes generation. However, the SOAP notes generated may be modified or edited by the Clinician 502, if there is any need to make changes in the planning.

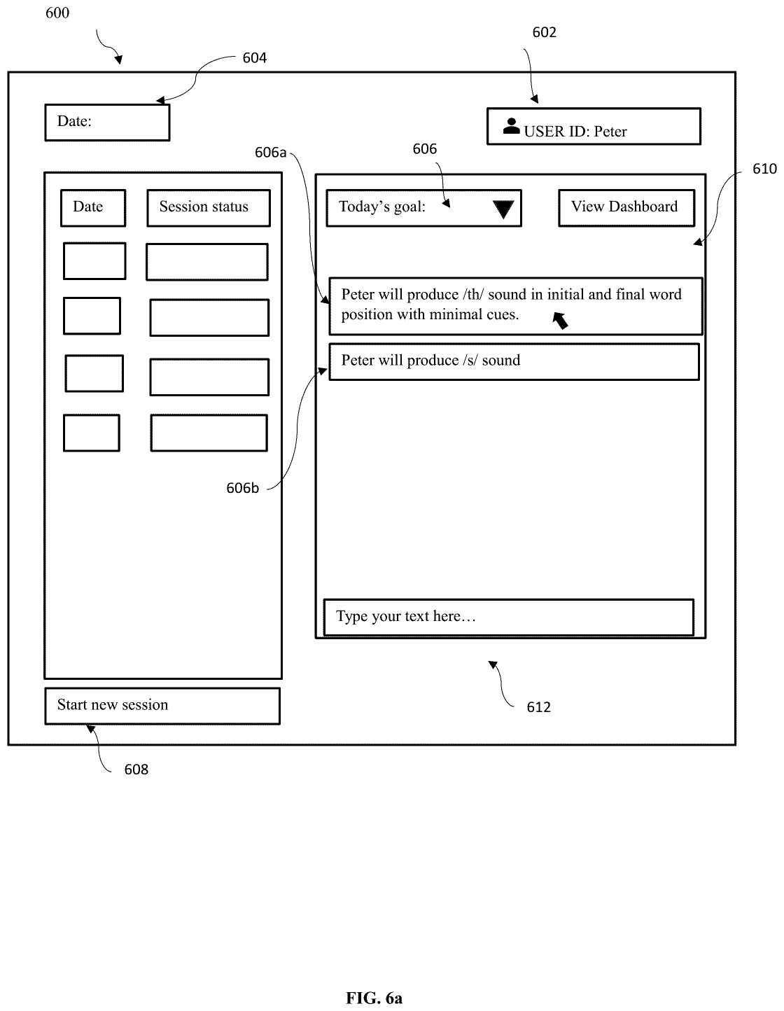

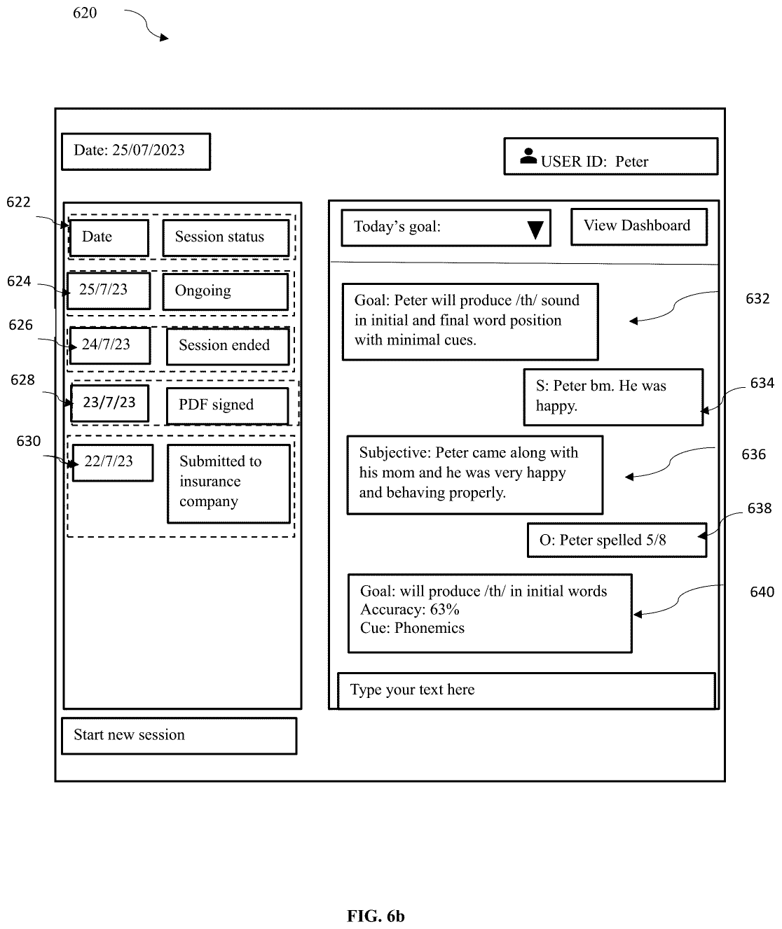

FIG. 6a illustrates an exemplary view of the dashboard 600 showing the user details and goal selection for automatically generating a medical session report of a patient, in accordance with one implementation of the present disclosure.

The exemplary view of dashboard 600 is shown in FIG. 6a which discloses the details when the Clinician starts the therapy session. It further discloses the basic structure of the dashboard.

The dashboard 600 contains a user ID tab 602 and date tab 604 at the top right and left corner of the dashboard 600 respectively. The user ID tab 602 contains the name or ID of the patient. The user ID allotted to each patient is unique and the whole details of the patients i.e., the SOAP notes generated in the past session, SOAP notes of ongoing session, medical details of patients, other details of patient and so on gets stored in that unique user ID. The date tab 604 depicts the date on which the current session/therapy of the patient is taking place.

The Clinician can start a new session by clicking on the tab 608 indicating ‘Start new session’. As soon as the new therapy session gets started, the Clinician gets an option at the top of the dashboard 600 which allows Clinician to select any specific goal for the ongoing session. The tab 606 indicates “Today's goal” which can be selected by the Clinician. The tab 606 consists of a drop-down menu which shows multiple goals that are designated for the patient. In an exemplary scenario, the tab 606a indicates a goal stating “Peter will produce /th/ sound in initial and final words position with minimal cues” and the tab 606b indicates a goal stating “Peter will produce /s/ sound” and so on. There can be any number of goals specified by the Clinician as per the treatment of the patient. The Clinician can select the specified target goal that he/she needs to focus in that particular therapy session. In this example, the Clinician selects the first goal (as shown in 606a) by clicking on it using mouse.

The dashboard 600 further contains a tab 612 which allows the Clinician to enter the patient's data in one or more format, which may include text, image, audio, video, recording or a combination of all, which are then converted in the form of detailed SOAP notes. The tab 612 allows Clinician to enter the input, text in case of present example, as it mentions “Type your text here . . . ”. Although the patient's data is not limited to text input, it can be provided by the Clinician in any form like audio, image, video or a combination of these. Using the same tab 612, the Clinician can provide input in various forms.

The tab 610 shows the option of “View dashboard” which allows Clinician to view the initial page of the dashboard 600 which contains all the information of the patient. The information may include past health records of patient, past generated SOAP notes, billing/insurance company status, other health details of the patient and so on.

FIG. 6b illustrates an exemplary view of the dashboard 620 showing the details inputted by the Clinician and details of SOAP notes prepared on different dates for automatically generating a medical session report of a patient, in accordance with one implementation of the present disclosure.

The exemplary view of dashboard 620 is shown in FIG. 6b which discloses the details when the Clinician starts the therapy session and starts inputting the patient's data based on the goal selected 632 for the patient for the ongoing session. It further discloses the details of various dates and status of the session on that specific date 622.

The Clinician starts the session by clicking on the tab “Start new session” and selects a goal for that particular session by clicking on the drop-down menu on the tab “Today's goal”. After selecting the goal, the selected goal appears at the top of the screen and the Clinician start inputting the patient's data based on the observation during the ongoing session with the patient. In an exemplary scenario, the Clinician initially inputs “S: Peter bm. He was happy” 634. The machine learning module generates an enhanced version of the input provided by the Clinician stating “Subjective: Peter came along with his mom. He was very happy and behaving properly” 636. The input provided by the Clinician may be written along with the prompt ‘S:’ or without the prompt ‘S:’. The contextual analysis module present in the data model of the system is smart enough to classify the input from the Clinician into its specific category i.e., Subjective, Objective, Assessment and Planning. Further, if the Clinician enters another input stating “O: Peter spelled 5/8” 638. The result would be generated by the machine learning module indicating the progress of the patient which in turn would be automatically converted to a detailed session note. The result 640 is declared in the following order:
“Goal: will produce /th/ in initial words
   Accuracy: 63%
   Cue: Phonemics”.
      This shows that the goal of the ongoing therapy is to make Peter pronounce /th/ in the initial word using a minimal number of cues, if needed and as a result, Peter's accuracy was 63% during the session.

It should be noted that the process of goal selection may be manual as well as automatic. The automatic goal selection allows Clinician to just focus on the therapy of the patient, rather than getting engaged in all the extra tasks which conventionally consumes a lot of time. The Clinician when provides patient's data as an input which may be further categorized in Subjective and Objective category of the SOAP notes using contextual analysis module. The machine learning module automatically selects the goal for the patient based on the input provided by the Clinician. The machine learning module then automatically maps the progress of the patient on the basis of inputs provided by the Clinician.

Further, there are various dates and session status 622 shown in the dashboard 620. These dates mention the details of the date at which the particular session was conducted and the session status discloses the current status of the session conducted on that particular date. In an exemplary scenario, as shown in the FIG. 6b, the tab 624 depicts that date as ‘25/7/2023’ and session status as ‘ongoing’ which means that these are the details of the current ongoing session with the patient. Rest other dates and session status (626, 628 and 630) are the past sessions conducted prior to ongoing session. The respective dates of all the sessions are mentioned. The session status includes session ended 626, PDF signed 628, submitted to insurance company 630 and so on. The session status ‘session ended’ 626 means that the session or therapy of the patient is over. The session status ‘PDF signed’ 628 means that the automatically generated SOAP note is reviewed by the Clinician and changes are also made, if needed and is further signed by the Clinician which can be further communicated to one or more remote users. The session status ‘submitted to the insurance company’ 630 means that the signed PDF form the Clinician is communicated with the insurance/billing companies to perform reimbursement to the patient in need.

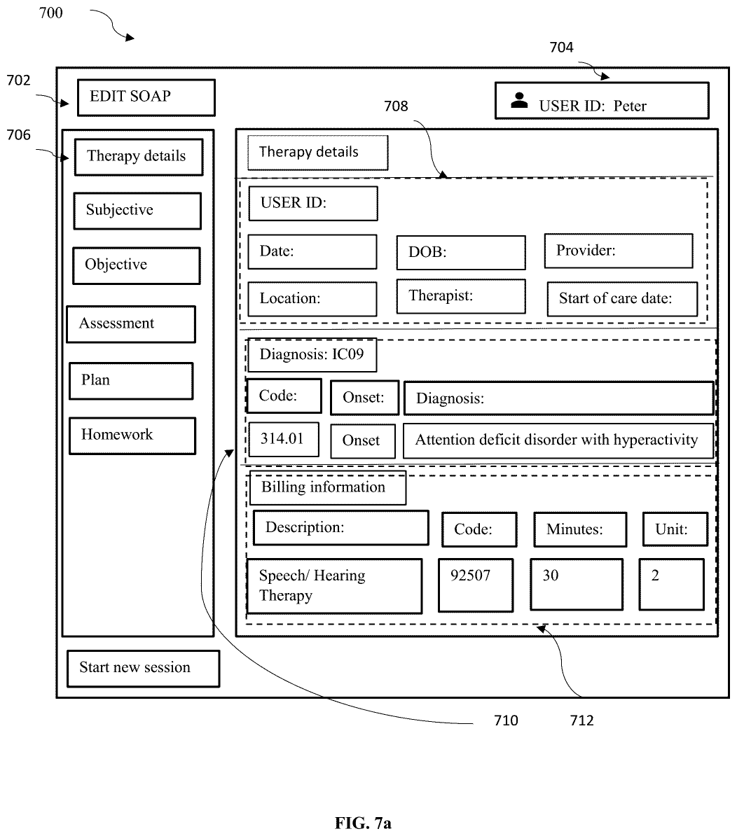

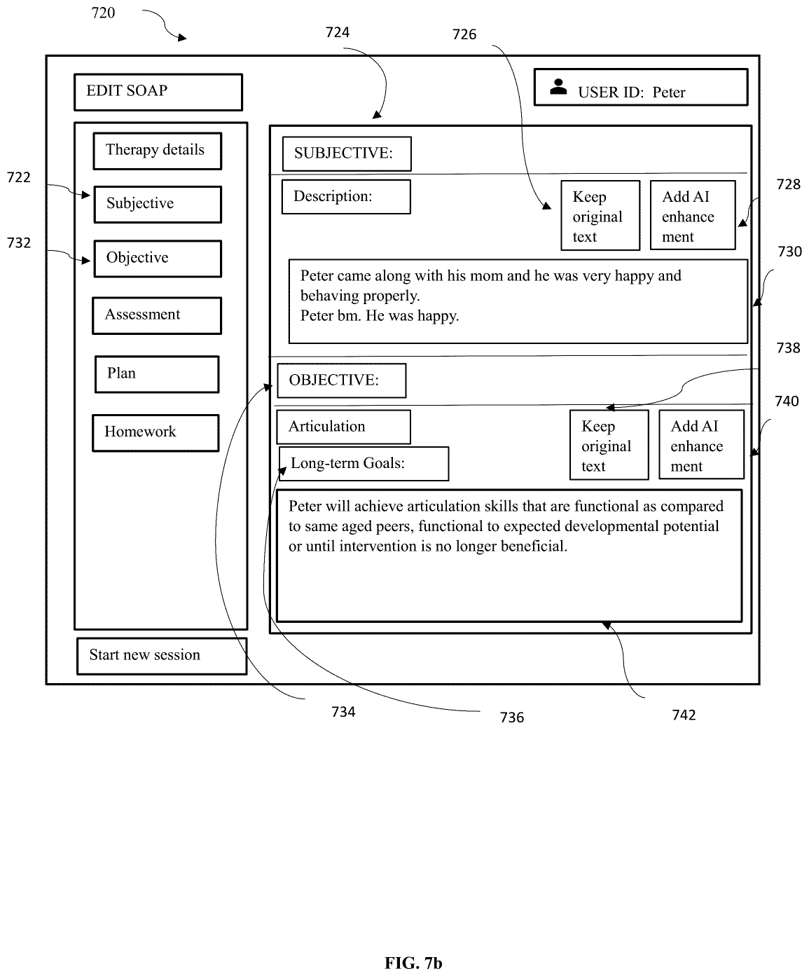

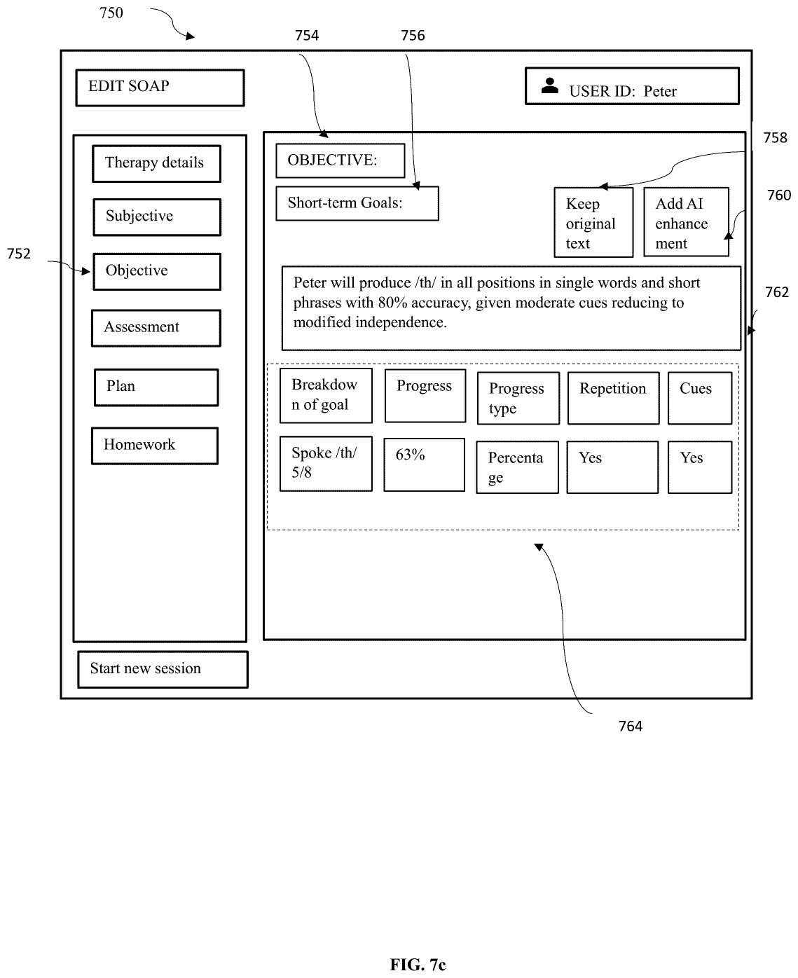

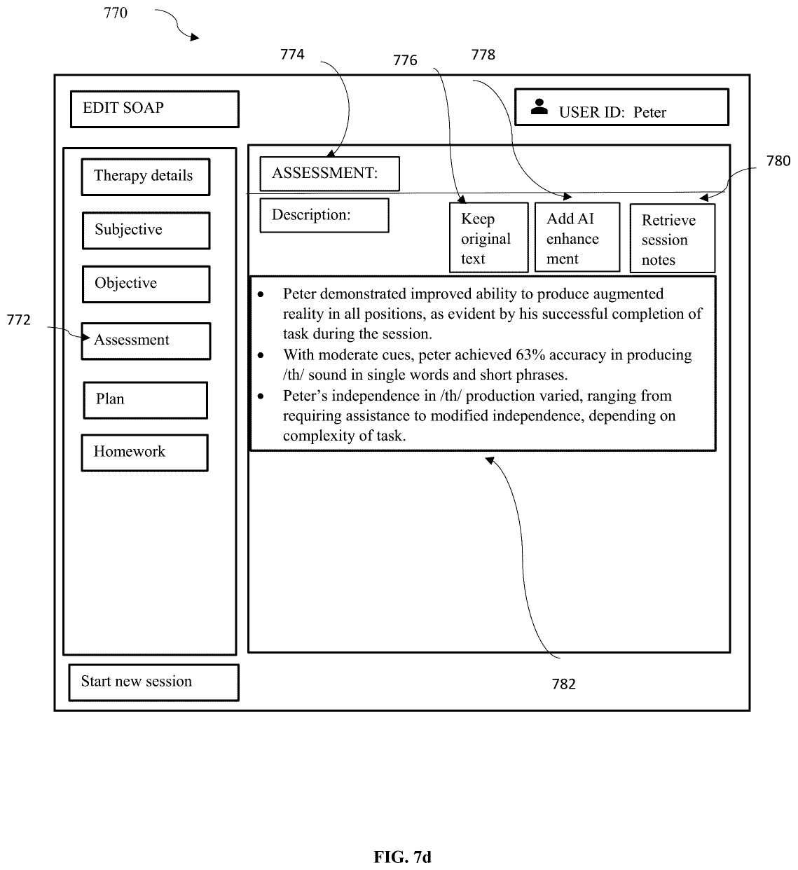

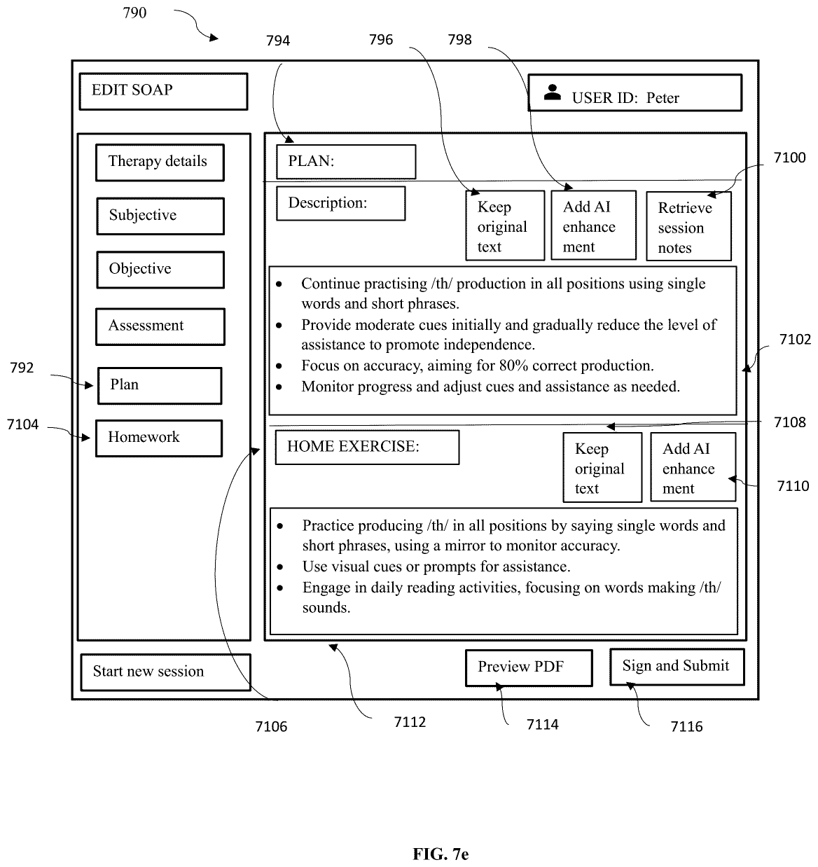

FIG. 7a illustrates an exemplary view of the dashboard 700 showing the process of editing the SOAP notes inputted by the Clinician on the dashboard 700 which mainly includes the therapy details and the billing information, in accordance with one implementation of the present disclosure.

The exemplary view of dashboard 700 is shown in FIG. 7a which discloses the details of the dashboard 700 when the SOAP notes are automatically generated using data model and the Clinician undergoes a review and makes modifications in the automatically generated SOAP notes, if needed. It further discloses the therapy details 708, diagnosis details 710 and billing information 712 for the billing/insurance companies.

After the Clinician finishes inputting the patient's data the data model of the system 100 comes into play. The data model presented in the system 100 has a machine learning module and a contextual analysis module which helps in automatic generation of the SOAP notes. The automatically generated SOAP notes are reviewed by the Clinician and can be further edited or modified by the Clinician, incase if it is needed. For this, the dashboard 700 offers a ‘Edit SOAP’ tab 702.

The User ID tab 704 is mentioned at top right corner of the dashboard 700. There are various categories available for the Clinician to review and edit, if needed. This includes Therapy details 706, Subjective, Objective, Assessment, Plan and Homework. In this FIG. 7a, the therapy details 706 section is explained in detail. The others will be explained in detail in the FIG. 7b-7e.

When the Clinician clicks on the tab ‘Therapy Details’ 706 and the tabs of ‘Therapy details’ 708, ‘Diagnostic’ details 710 and ‘Billing details’ 710 starts appearing. The ‘Therapy details’ 708 further includes details such as user ID of the patient, date of the ongoing session, date of birth of the patient, location of the therapy center, name of the therapist i.e., the Clinician who is taking the sessions or therapy, name of the provider i.e., the person with whom the child or patient came to the therapy center and the start of the care date i.e., the initial date when the child has started his/her therapy. These details may be filled by the Clinician or may be automatically recovered from the past generated SOAP notes or the one or more session notes of the ongoing session. In an example, the date and provider name can be filled up by the machine learning module using the details of ongoing session (as explained above in detail) and the other details can be retrieved from the past generated SOAP notes. In case, if any correction is needed, for instance, change in the name of the therapist and so on the Clinician may edit the particular field.

The dashboard 700 further includes the ‘diagnostic’ details 710 which are automatically filled based on the one or more session notes of the ongoing session and the past generated SOAP notes. The tab of ‘Diagnostic’ details is provided a code for instance, ‘IC09’. These codes are defined by the AMA (American Medical Association) where each condition is given a unique code. The code has a specific meaning and this particular code, IC09 is used for treatment of ‘Attention deficit disorder with hyperactivity’. The SOAP notes are prepared using these codes details to maintain consistency of treatment. The tab of ‘Diagnostic’ details 710 further includes the code number, onset and the diagnosis which explains the code in brief, for instance, in the given example, the code allotted to the diagnosed condition is ‘314.01’ which indicates the diagnosis of ‘Attention deficit disorder with hyperactivity’ mentioned in the diagnosis details.

The dashboard 700 further includes ‘Billing information’ 712, which is further used by billing/insurance companies for making reimbursement to the patient in need. It further contains details like ‘description of the therapy’ that the patient is undergoing, ‘code name’ of the therapy which is uniquely allotted to each patient, ‘timing of the therapy’, ‘number of units’ which means that till that time how many sessions have been conducted. All these information is used by the billing/insurance companies while issuing the reimbursement to the patient, for instance, in the given example, the patient is undergoing ‘Speech/Hearing therapy’ which is mentioned in the description and the code allotted to the therapy is ‘92507’. Further, till the time the patient has undergone 2 therapies, so the units are mentioned as 2 and the timing for each therapy is 30 minutes, so in place of timing 30 minutes is mentioned.

FIG. 7b illustrates an exemplary view of the dashboard 720 showing the process of editing the SOAP notes inputted by the Clinician on the dashboard 720 which mainly includes the subjective and objective aspect (long-term goals) of the SOAP notes, in accordance with one implementation of the present disclosure.

The exemplary view of dashboard 720 is shown in FIG. 7b which discloses the details of the dashboard 720 when the SOAP notes are automatically generated using data model and the Clinician undergoes a review and makes modifications in the automatically generated SOAP notes, if needed. It further discloses the details of the Subjective 722 and Objective 732 category of the SOAP notes.

The User TD tab is mentioned at top right corner of the dashboard 720. There are various categories available for the Clinician to review and edit, if needed. This includes Therapy details, Subjective 722, Objective 732, Assessment, Plan and Homework. In this Figure, the Subjective 722 and Objective 732 section is explained in detail. The others will be explained in detail in the FIG. 7c-7e.

When the Clinician clicks on the Subjective category 722 of the SOAP notes, the dashboard 720 shows the details of the Subjective category 724 of the respective user ID. The ‘Subjective’ category 724 further contains some other tabs like ‘Keep original text’ 726, ‘Add AI enhancement’ 728 and ‘Description’ 730 of the Subjective tab 722 of the SOAP notes. The feature ‘Keep original text’ 726 allows the Clinician to keep the input as it was, as entered by the Clinician. On the other hand, the feature ‘Add AI enhancement’ 728 allows Clinician to use the enhanced and detailed version of the one or more session notes. This detailed version is provided by using machine learning techniques and large language model. The ‘Description’ 730 provides the detailed enhanced result of the Subjective category 724 in the form of SOAP notes.

In an exemplary scenario, in accordance with the FIGS. 6a and 6b, the Clinician input patient's data. The machine learning module and the contextual analysis module classifies them and processes them to automatically generate SOAP notes by compiling all the generated session notes. The output generated by the data model states that:
“Subjective: Peter came along with his mom and he was very happy and behaving properly.
Peter bm. He was happy.”
   Both the results are produced i.e., the original text (text in the case of present example, although it could be image, audio, video, recording and so on) inputted by the Clinician and the machine generated enhanced and detailed version of the SOAP note. The Clinician can include either both of them or can select any one of them. The Clinician may further make changes in the machine generated SOAP notes, if needed.

Similarly, the Clinician when click on the ‘Objective’ tab 732 of the SOAP notes, the dashboard 720 displays the ‘Objective’ category 734 of the SOAP notes. The ‘Objective’ category 734 of the SOAP notes further contains some other tabs like ‘Keep original text’ 738, ‘Add AI enhancement’ 740 and ‘Long-term goals’ 742 of the Objective tab 734 of the SOAP notes. The feature ‘Keep original text’ 738 allows the Clinician to keep the input as it was, as entered by the Clinician. On the other hand, the feature ‘Add AI enhancement’ 740 allows Clinician to use the enhanced and detailed version of the one or more session notes. This detailed version is provided by using machine learning techniques and large language model. The ‘Long-term goals’ 742 provides the detailed enhanced result of the long-term goals selected for the treatment of the patient under Objective category 734 in the form of SOAP notes.

In an exemplary scenario, in accordance with the FIGS. 6a and 6b, the at least the Clinician input patient's data. The machine learning module and the contextual analysis module classifies them and processes them to automatically generate SOAP notes by compiling all the generated session notes. The output 736 generated by the data model states that “Objective: Peter will achieve articulation skills that are functional as compared to same aged peers, functional to expected developmental potential or until intervention is no longer beneficial”. The machine generated enhanced and detailed version of the SOAP note are produced as a result of the Clinician input. The Clinician may further make changes in the machine generated SOAP notes, if needed.

FIG. 7c illustrates an exemplary view of the dashboard 750 showing the process of editing the SOAP notes inputted by the Clinician on the dashboard 750 which mainly includes the Objective aspect (short-term goals) of the SOAP notes, in accordance with one implementation of the present disclosure.

The exemplary view of dashboard 750 is shown in FIG. 7c which discloses the details of the dashboard 750 when the SOAP notes are automatically generated using data model and the Clinician undergoes a review and makes modifications in the automatically generated SOAP notes, if needed. It further discloses the details of the Objective 752 category of the SOAP notes.

The User ID tab is mentioned at top right corner of the dashboard 750. There are various categories available for the Clinician to review and edit, if needed. This includes Therapy details, Subjective, Objective 752, Assessment, Plan and Homework. In this Figure, the Objective 752 section is explained in detail. The others will be explained in detail in the FIG. 7d-7e.

The Clinician when click on the ‘Objective’ tab 752 of the SOAP notes, the dashboard 750 displays the ‘Objective’ category 754 of the SOAP notes. The ‘Objective’ category 754 of the SOAP notes further contains some other tabs like ‘Keep original text’ 758, ‘Add AI enhancement’ 760 and ‘Short-term goals’ 756 of the Objective tab 754 of the SOAP notes. The feature ‘Keep original text’ 758 allows the Clinician to keep the input as it was, as entered by the Clinician. On the other hand, the feature ‘Add AI enhancement’ 760 allows Clinician to use the enhanced and detailed version of the patient's data. This detailed version is provided by using machine learning techniques and large language model. The ‘Short-term goals’ 756 provides the detailed enhanced result of the short-term goals selected for the treatment of the patient under Objective category 754 in the form of SOAP notes.

In an exemplary scenario, in accordance with the FIGS. 6a and 6b, the at least the Clinician input patient's data. The machine learning module and the contextual analysis module classifies them and processes them to automatically generate SOAP notes by compiling all the generated progress notes. The output 762 generated by the data model states that:
“Objective: Peter will produce /th/ in all positions in single words and short phrases with 80% accuracy, given moderate cues reducing to modified independence.”.
   The machine generated enhanced and detailed version of the SOAP note are produced as a result of the Clinician input. The Clinician may further make changes in the machine generated SOAP notes, if needed.

Further, the dashboard 750 shows the report of the patient's progress under Objective category 754. It includes ‘breakdown of the goal’, ‘progress status in terms of percentage’, ‘repetition and cues status’ i.e., whether cues are provided to the child during therapy or not, for instance, in case of present example, the breakdown of the goal indicates ‘spoke /th/ 5/8’. The progress type is calculated in terms of percentage which is 63% in case of present example. Further, the details of repetition made and cues provided to the patient during the therapy is provided. The repetition is made which is answered by ‘yes’ and the cues are also provided to the child during therapy, hence a ‘yes’ is mentioned there in the response.

FIG. 7d illustrates an exemplary view of the dashboard 770 showing the process of editing the SOAP notes inputted by the Clinician on the dashboard 770 which mainly includes the assessment aspect of the SOAP notes, in accordance with one implementation of the present disclosure.

The exemplary view of dashboard 770 is shown in FIG. 7c which discloses the details of the dashboard 770 when the SOAP notes are automatically generated using data model and the Clinician undergoes a review and makes modifications in the automatically generated SOAP notes, if needed. It further discloses the details of the Assessment 772 category of the SOAP notes.

The User ID tab is mentioned at top right corner of the dashboard 770. There are various categories available for the Clinician to review and edit, if needed. This includes Therapy details, Subjective, Objective, Assessment 772, Plan and Homework. In this Figure, the Assessment 772 section is explained in detail. The others will be explained in detail in the FIG. 7e.

The Clinician when click on the ‘Assessment’ tab 772 of the SOAP notes, the dashboard 770 displays the ‘Assessment’ category 774 of the SOAP notes. The ‘Assessment’ category 774 of the SOAP notes further contains some other tabs like ‘Keep original text’ 776, ‘Add AI enhancement’ 778 and ‘Retrieve progress notes’ 780 of the Assessment tab 774 of the SOAP notes. The feature ‘Keep original text’ 776 allows the Clinician to keep the input as it was, as entered by the Clinician. On the other hand, the feature ‘Add AI enhancement’ 778 allows Clinician to use the enhanced and detailed version of the session notes. This detailed version is provided by using machine learning techniques and large language model. The feature ‘Retrieve session notes’ 780 allows Clinician to retrieve the session notes generated in the previous session as it was. Since, in therapy sessions like speech therapy, behavioral therapy and so on the assessment and planning for the child does not changes after a single therapy. There are chances that the same therapy and its associated planning need to be continued for next several therapies. The patient/therapist may use this feature to retrieve the past generated session notes in the ongoing session. This will save the time and the Clinician can focus on the therapy sessions of the child.

In an exemplary scenario, in accordance with the FIGS. 6a and 6b, the at least the Clinician input patient's data. The machine learning module and the contextual analysis module classifies them and processes them to automatically generate SOAP notes by compiling all the generated session notes. The output 782 generated by the data model states that
“Assessment: Peter demonstrated improved ability to produce augmented reality in all positions, as evident by his successful completion of task during the session.
With moderate cues, peter achieved 63% accuracy in producing /th/ sound in single words and short phrases.
Peter's independence in /th/ production varied, ranging from requiring assistance to modified independence, depending on complexity of task”.
   The machine generated enhanced and detailed version of the SOAP note are produced as a result of the Clinician input. The Clinician may further make changes in the machine generated SOAP notes, if needed.

FIG. 7e illustrates an exemplary view of the dashboard 790 showing the process of editing the SOAP notes inputted by the Clinician on the dashboard 790 which mainly includes the planning and home-based exercise aspect of the SOAP notes, in accordance with one implementation of the present disclosure.

The exemplary view of dashboard 790 is shown in FIG. 7e which discloses the details of the dashboard 790 when the SOAP notes are automatically generated using data model and the Clinician undergoes a review and makes modifications in the automatically generated SOAP notes, if needed. It further discloses the details of the Plan 792 and Homework 7104 category of the SOAP notes.

The User ID tab is mentioned at top right corner of the dashboard 790. There are various categories available for the Clinician to review and edit, if needed. This includes Therapy details, Subjective, Objective, Assessment, Plan 792 and Homework 7104. In this Figure, the Plan 792 and Homework 7104 section is explained in detail.

The Clinician, when clicks on the Plan category 792 of the SOAP notes, the dashboard 790 shows the details of the Plan category 794 of the respective user ID. The ‘Plan’ category 794 further contains some other tabs like ‘Keep original text’ 796, ‘Add AI enhancement’ 798 and ‘Retrieve progress notes 7100 of the Plan tab 792 of the SOAP notes. The feature ‘Keep original text’ 796 allows the clinician to keep the input as it was, as entered by the clinician. On the other hand, the feature ‘Add AI enhancement’ 798 allows Clinician to use the enhanced and detailed version of the patient's data. This detailed version is provided by using machine learning techniques and large language model. The feature ‘Retrieve session notes 7100 allows Clinician to retrieve the session notes generated in the previous session as it was. Since, in therapy sessions like speech therapy, behavioral therapy and so on the assessment and planning for the child does not changes after a single therapy. There are chances that the same therapy and its associated planning need to be continued for next several therapies. The patient/therapist may use this feature to retrieve the past generated session notes in the ongoing session. This will save the time and the Clinician can focus on the therapy sessions of the child.

In an exemplary scenario, in accordance with the FIGS. 6a and 6b, the at least the Clinician input patient's data. The machine learning module and the contextual analysis module classifies them and processes them to automatically generate SOAP notes by compiling all the generated session notes. The output 7102 generated by the data model states that:
“Plan: Continue practicing /th/ production in all positions using single words and short phrases.
Provide moderate cues initially and gradually reduce the level of assistance to promote independence.
Focus on accuracy, aiming for 80% correct production.
Monitor progress and adjust cues and assistance as needed.”

The machine generated enhanced and detailed version of the SOAP note are produced as a result of the Clinician input. The Clinician may further make changes in the machine generated SOAP notes, if needed.

Similarly, the Clinician when click on the ‘Homework’ tab 7104 of the SOAP notes, the dashboard 790 displays the ‘Home Exercise’ category 7106 of the SOAP notes. The ‘Home Exercise’ category 7106 of the SOAP notes further contains some other tabs like ‘Keep original text’ 7108, ‘Add AI enhancement’ 7110 of the Objective tab 734 of the SOAP notes. The feature ‘Keep original text’ 7108 allows the Clinician to keep the input as it was, as entered by the Clinician. On the other hand, the feature ‘Add AI enhancement’ 7110 allows Clinician to use the enhanced and detailed version of the patient's data. This detailed version is provided by using machine learning techniques and large language model. The details 7112 of the ‘Home Exercise’ category 7106 is provided to the parents/caregivers so that they can provide the same kind of therapy to the child at home also. This will enhance the progress of the child during the treatment. The Home Exercise can also be provided to the parents/caregivers in the form of videos that would guide them how to perform the therapy procedure at home.

In an exemplary scenario, in accordance with the FIGS. 6a and 6b, the at least the Clinician input patient's data. The machine learning module and the contextual analysis module classifies them and processes them to automatically generate SOAP notes by compiling all the generated session notes. The output 7112 generated by the data model states that
“Home exercise: Practice producing /th/ in all positions by saying single words and short phrases, using a mirror to monitor accuracy.
Use visual cues or prompts for assistance.
Engage in daily reading activities, focusing on words making /th/ sounds”.
   The machine generated enhanced and detailed version of the SOAP note are produced as a result of the Clinician input. The Clinician may further make changes in the machine generated SOAP notes, if needed.

The dashboard 790 further contains two tabs namely ‘Preview PDF’ 7114 and “Sign and Submit’ 7116. The tab ‘Preview PDF’ 7114 helps the Clinician to review the automatically generated SOAP note, which is in PDF format. The tab “Sign and Submit’ 7116 allows Clinician to sign and submit the automatically generated SOAP notes. After this process, the automatically generated SOAP notes are communicated to one or more remote users, which may include insurance/billing companies, parents/caregivers, another doctors/physician related to same field and so on.

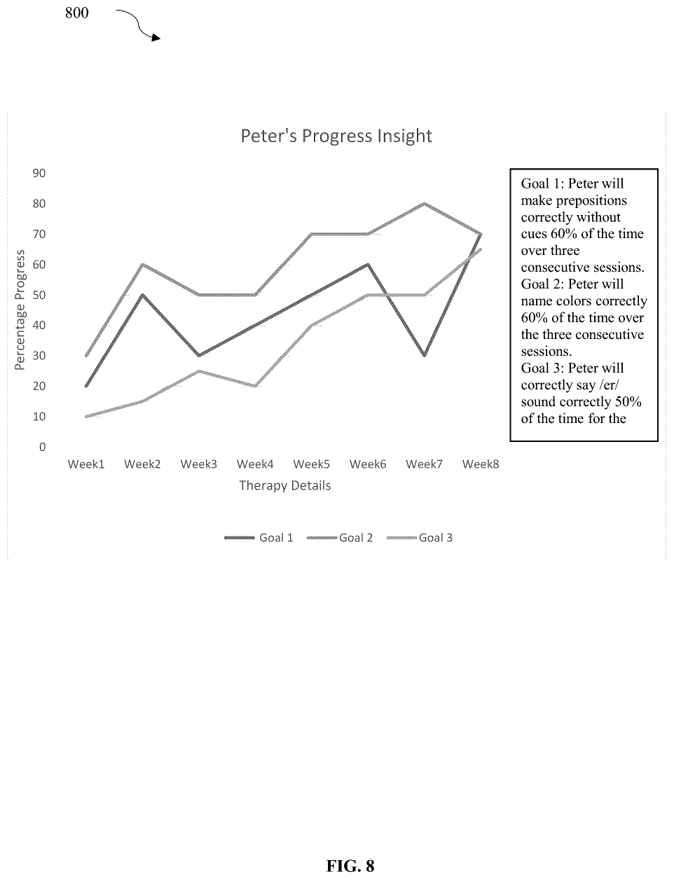

FIG. 8 illustrates an exemplary graphical representation 800 of the progress made by the patient after a few therapy sessions, in accordance with one implementation of the present disclosure.

The graph 800 shows the visual representation of the progress made by the patient in the form of a line graph. However, it could be in any other format such as a pie chart, area chart, bubble chart, or a combination thereof. This graph 800 can be accessed by the one or more remote users using the disclosed platform. It makes it easier for the one or more remote users to understand the progress made by the patient after one or more sessions i.e., whether the patient is showing any positive or negative response with respect to the therapy sessions provided to him/her. It also saves a lot of time of the Clinician, which can be further utilized for the therapy session.

The graph 800 shows progress made by a patient named Peter against 3 goals after 8 sessions, where he had one session per week. As shown, the graph 800 includes an X-axis depicting “Therapy Details” and Y-axis depicting “Percentage Progress” made by Peter against 3 different goals allotted to him automatically using data model. The goals include: “Goal 1—Peter will make prepositions correctly without cues 60% of the time over three consecutive sessions.”, “Goal 2—Peter will name colors correctly 60% of the time over the three consecutive sessions.”, and “Goal 3—Peter will correctly say /er/ sound correctly 50% of the time over three consecutive sessions.”

Here, in this example, Peter is undergoing a speech therapy session and has been allotted one or more long-term and short-term goals automatically using data model. Based on the automatically generated session notes, the data model analyzes and prepares a graph 800 which indicates the progress made by Peter after the 8-therapy session that spanned across 8 consecutive weeks. The graph 800 shows the percentage progress of the Peter based on the 3 goals decided for him on a weekly basis.

Although this is just an exemplary scenario showing the graph 800 on a per therapy basis. There could be many more cases like monthly, quarterly, yearly progress graphs of the child, graph related to a single goal, and so on.

Further, the Clinician may generate a progress report after a specific interval of time, say 3 months. This progress report is a consolidated form of the medical session report of all the therapy sessions that the patient has undergone during that time interval i.e., 3 months in case of present example. The Clinician may communicate the progress report to the insurance/billing companies, parents/caregivers, other physicians/doctors and so on. The Clinician may also keep the progress record with himself/herself in order to check the progress of the patient undergoing therapy. The time interval for generating the progress report may vary and depends on the state at which the patient is responding to a therapy or the requirements of the parents/caregivers and/or insurance/billing companies to know the ongoing health status of the patient. It should be noted that the progress report may consider other medical records of the patient and session notes form previous sessions.

The system also allows the automatic generation of the discharge summary report based on the patient's data inputted by a Clinician in one or more formats. This scenario may occur under the circumstances where the patient's treatment is completed and he/she has recovered to a great extent from the specific health condition or when the patient switches his/her Clinician, which may be due to any reason like relocation of the patient, negative response from the therapy of the Clinician, occurrence of any new greatly qualified clinician in the locality and so on. The patient when terminates the therapy his/her Clinician provides a discharge summary, which is automatically generated in the present disclosure. The discharge summary is generated based on the overall progress made by the patient across various allocated goals during the entire course of therapy. Once the discharge summary is generated, the summary is uploaded on the platform where it can be accessed by the one or more remote users like parents and/or caregivers of the patient, Clinician and so on.

As described throughout this application the term “dashboard” is used to mean a system, application, or any other similar software platform. Also, the terms “parents”, “caregivers”, “users” or “members” will have the similar meaning throughout the disclosure. In addition, the system of the present disclosure is relatively inexpensive, safe and easy to use.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

### TECHNICAL ADVANCEMENTS

The present disclosure described herein above for automatically generating a medical session report of condition in patients has several technical advantages including, but not limited to, the realization of:
automatically generates SOAP notes without manual intervention;
reduces time of the Clinician in documentation;
suitable for predicting the progress of health condition;
user friendly;
easy to use;
versatility of the user interface;
automatic selection of goals and updating the progress status of the child;
visual representation of the progress of the child in the form of graphs, charts which makes it easier for parents, therapist to understand the progress status without reading the long summary;
maintains transparency and security of the data.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

We claim:

1. A method for automatically generating a medical session report for one or more sessions of a patient with a Clinician, the method comprising:
receiving a patient's data in one or more formats from one or more sources;
processing the patient's data using machine learning techniques and language learning models by extracting relevant information from the patient's data;
generating one or more session notes automatically based on the patient's data, the processed patient's data, and one or more pre-stored information; and
compiling the one or more session notes to automatically generate the medical session report of the patient.

2. The method for automatically generating a medical session report as claimed in claim 1, wherein the medical session report of the patient includes a SOAP note, wherein the SOAP note includes details related to subjective information, objective observations, assessment findings, and plans for future treatment of the patient.

3. The method for automatically generating a medical session report as claimed in claim 1, wherein the one or more sources may include Clinician's short-hand notes from the session, audio recording of the session, video recording of the session, transcript of the session, patient's past electronic health records, past session notes, clinical assessments, patient's interviews and other relevant health condition information.

4. The method for automatically generating a medical session report as claimed in claim 1, wherein the patient's data may be in one or more of the following formats—text, audio, video, or a combination thereof.

5. The method for automatically generating a medical session report as claimed in claim 1, wherein generating the one or more session notes further includes populating the patient's data with the one or more pre-stored information, wherein the one or more pre-stored information includes one or more previously stored patient's data, another patient's data having similar manifestation of the condition and/or information obtained from a database.

6. The method for automatically generating a medical session report as claimed in claim 1, wherein the at least one or more session notes are edited by the Clinician.

7. The method for automatically generating a medical session report as claimed in claim 1, wherein the machine learning techniques includes a machine learning algorithm or a natural language processing (NLP) algorithm and the large language models include one or more generative AI models either in combination or alone.

8. The method as claimed in claim 1, further comprises:
selecting a template in correspondence to the generated one or more session notes of the patient, wherein a template is selected from one or more pre-stored templates;
compiling the one or more session notes in the selected template to generate the medical session report of the patient.

9. The method for automatically generating a medical session report as claimed in claim 1, wherein the patient's past electronic health records is analysed and adapted in the at least one or more session notes in order to enhance the specificity and relevance of the generated session notes.

10. The method for automatically generating a medical session report as claimed in claim 1, wherein an Insurance Claim is automatically generated based on the patient's medical session report.

11. The method for automatically generating a medical session report as claimed in claim 1, wherein the medical session report is communicated to one or more remote users including one or more insurance companies, caregivers of the patient and other Clinicians.

12. A system for automatically generating a medical session report for one or more sessions of a patient with a Clinician, the system comprising:
a server including a memory to store various treatment plans and a patient's previous and ongoing medical data; and
a processing device operatively coupled to the server, wherein the processing device is configured to execute the instructions to:
receive a patient's data in one or more formats from one or more sources;
process the patient's data using machine learning techniques and language learning models by extracting relevant information from the patient's data;
generate one or more session notes automatically based on the patient's data, the processed patient's data, and one or more pre-stored information; and
compile the one or more session notes to automatically generate the medical session report of the patient.

13. The system as claimed in claim 12, further includes a data model configured to perform a contextual analysis on the received patient's data and based on the contextual analysis, one or more goals related to the condition of the patient are automatically selected.

14. The system as claimed in claim 13, wherein the data model may be a machine learning model configured to process and analyse the patient's data inputted by the Clinician using techniques such as convolutional neural networks, audio signal processing, or video signal processing.

15. The system as claimed in claim 13, wherein the data model uses Generative AI model which may include any one of the GPT-3 (Generative Pre-trained Transformer 3), GPT-3.5, BERT (Bidirectional Encoder Representations from Transformers), RoBERTa (Robustly Optimized BERT Approach), ALBERT (A Lite BERT), T5 (Text-to-Text Transfer Transformer) or a combination thereof.

16. The system as claimed in claim 12, wherein the patient's data may include past session notes made by the Clinician, ongoing session notes, patient's past electronic medical records, treatment plans and a combination thereof.

17. The system as claimed in claim 12 includes one or more user interfaces including a graphical user interface, a voice user interface, a video user interface, a gesture user interface, or a combination thereof.

18. The system as claimed in claim 12 automatically updates one or more goals for the patient based on the patient's data, wherein the updates include marking one or more previously assigned goals as complete, assigning new goals, and mapping of goals against the patient's data or inputs provided by the Clinician.

19. The system as claimed in claim 18, wherein the system is configured to automatically generate one or more progress reports corresponding to the progress made by the patient over a period, wherein the progress reports are generated based on one or more medical records of the patient, previous session notes, previous medical session reports, or a combination thereof.

20. The system as claimed in claim 12 comprises providing one or more insights related to the progress made by the patient on one or more goals in the form of an infographic such as charts, graphs, tables in combination or alone, wherein the infographics are dynamic and a user interacts with the infographic to obtain one or more insights of interest.
